(19) United States
(12) Plant Patent Application Publication
(10) Pub. No.: US 2013/0340132 P1
(43) Pub. Date: Dec. 19, 2013

(54) Grapevine 'IFG Eleven'

(50) ;

(71) Applicant: David Cain, Bakersfield, CA (US)

(72) Inventor: David Cain, Bakersfield, CA (US)

(73) Assignee: International Fruit Genetics, LLC, Bakersfield, CA (US)

(21) Appl. No.: 13/507,265

(22) Filed: Jun. 18, 2012

(51) Int. Cl. A01H 5/00

(52) U.S. Cl. PLT/207

(57) ABSTRACT

This invention is a new and distinctive grapevine ‘IFG Eleven’ ‘IFG Eleven’ produces naturally large, elongated, crisp white seedless berries that require little or no exogenous application of gibberellic acid to obtain commercially acceptable berry size and ripen in mid to late season

### LATIN NAME OF THE GENUS AND SPECIES CLAIMED

Vitis vinifera

### VARIETY DENOMINATION

‘IFG Eleven’

### BACKGROUND OF THE INVENTION

The new and distinct grapevine described and claimed herein originated from a hand pollinated cross of the IFG 02013-090-033, an unnamed seedless selection from the IFG breeding program and the IFG 01034-069-026 another unnamed seedless selection from the IFG breeding program hybridized in May 2005. The abortive seed traces were subsequently embryo cultured and the resulting plant was planted in the field in April 2006. The present variety of grapevine was selected as a single plant in September 2007 and was first asexually propagated by hardwood cuttings in December 2007. The resulting propagules were planted during April 2008 near Delano, Kern County, Calif. and were found to reproduce true-to-type through at least two generations of asexual reproduction.

### BRIEF SUMMARY OF THE INVENTION

The new grapevine ‘IFG Eleven’ is characterized by producing naturally large, narrow elliptic, seedless berries having very small residual seed traces. Berries are firm, crisp in texture and ripen in mid to late season. Fruits normally ripen in early September near Delano, Calif.

To the inventor's knowledge, the known variety which the new grapevine variety is most similar to is the Thompson Seedless variety. ‘IFG Eleven’ can be distinguished from the Thompson Seedless variety by ripening almost one month later. The flesh of the ‘IFG Eleven’ is crisper than the flesh of the Thompson Seedless variety. ‘IFG Eleven’ does not require applications of gibberellin to thin clusters or size berries to obtain commercially acceptable berry size as is required for the Thompson Seedless variety. The ‘IFG Eleven’ has lower acidity than the Thompson Seedless variety. The ‘IFG Eleven’ is much more productive than the Thompson Seedless variety and can be spur pruned while Thompson Seedless requires cane pruning to attain commercially acceptable yields.

### BRIEF DESCRIPTION OF THE FIGURE

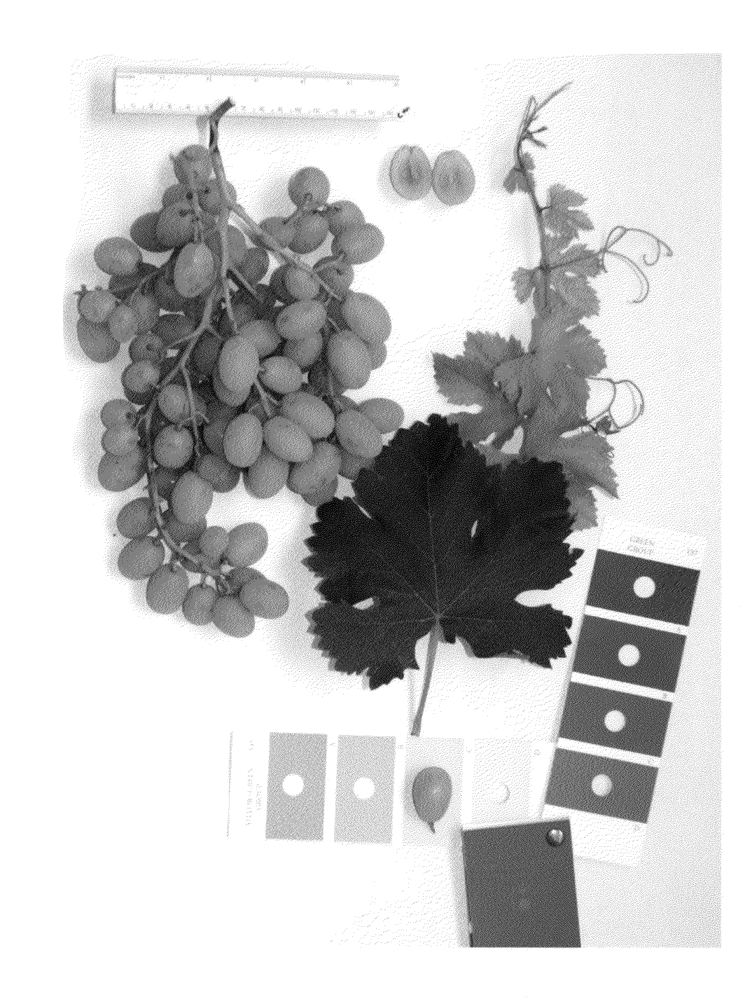

The accompanying photographic illustration in FIG. 1 illustrates in full color ‘IFG Eleven’. The colors are as nearly true as is reasonably possible in a color representation of this type.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published by The Royal Horticultural Society, London, England.

Throughout this specification subjective description values conform to those set forth by the International Plant Genetic Resources Institute publication ‘Descriptors for Grape’ (Vitis spp.) (1983) which was developed in collaboration with the Office International de la Vigne et du Vin (OIV) and the International Union for the Protection of New Varieties of Plants (UPOV).

The descriptive matter which follows pertains to ‘IFG Eleven’ plants grown in the vicinity of Delano, Kern County, Calif. during 2010 , and is believed to apply to plants of the variety grown under similar conditions of soil and climate elsewhere:
Vine:
   General.—Size — Medium Vigor — Medium to Weak Density of foliage — Medium to Weak Productivity — Very productive Root stock — Own root Training method — Typically spur pruned leaving 2 bud spurs.
   Trunk.—Trunk diameter of 4-year-old vines at 30 cm above the soil line — 5.5 cm Shape — Medium Straps — Short — Split Surface texture — Medium rough Inner bark color — Yellow-Brown; 164A, 165A, B.
Shoots:
   Young shoot.—Form of tip — Fully opened Distribution of anthocyanin coloration of tip — Absent Intensity of anthocyanin coloration of tip — Absent Density of prostrate hairs of tip — Sparse Density of erect hairs of tip — Absent Color — Yellow-Green; 144A, 146A, B, C.
   Woody shoots (mature canes).—Shape — Medium Internode length — Short; About 8.5 cm Width at node — About 0.9 cm Cross section — Circular Surface — Striate Main color — Yellowish brown; 174B, 177A, B, C Density of erect hairs of nodes — None Density of erect hairs on internodes — None Growth of axillary shoots — Medium; Approximately 26.2 cm.
   Flowering shoot.—Vigor during flowering — Strong Attitude during flowering on shoots not tied — Semi-erect Color — dorsal side of internodes — Green with Red stripes Color — ventral side of internodes — Green Color — dorsal side of nodes — Green Color — ventral side of nodes — Green Density of prostrate hairs of nodes — Very sparse Density of erect hairs of nodes — None Density of prostrate hairs on internode — Very sparse Density of erect hairs on internode — None Anthocyanin coloration of buds — Absent.
   Tendrils.—Distribution on the shoot (at full flowering) — Discontinuous Length of Tendril — Medium; About 22.4 cm Thickness — Medium Color — Yellow-Green; N144A, 152C, 151A Form — mostly Trifurcated and Quadfurcated Number of consecutive tendrils — 2.
Leaves:
   Young leaves.—Color of upper surface of first four distal unfolded leaves — Green Average intensity of anthocyanin coloration of six distal leaves prior to flowering — Absent or very weak Density of prostrate hairs between veins (lower surface) — Absent — Very sparse Density of prostrate hairs on veins (lower surface) — Very sparse Density of erect hairs between veins (lower surface) — Absent Density of erect hairs on veins (lower surface) — Very sparse.
   Mature leaves.—Average length — About 13.4 cm Average width — About 17.5 cm Mature leaf size — Large Shape of blade — Wedge-shaped Number of lobes — 5 Anthocyanin coloration of main veins on upper side of blade — Absent Mature leaf profile — Undulate Blistering surface of blade upper surface — Medium Leaf blade tip — In the plane of the leaf Undulation of margin — Slight Thickness — Medium Undulation of blade between main and lateral veins — Overall Shape of teeth — Mixture of both sides straight and both sides convex Length of teeth — Medium Ratio length/width of teeth — Small Shape of upper lateral sinuses — Lobes slightly overlapping — strongly overlapping Depth of upper lateral sinuses — Deep General shape petiole sinus — Half open Shape of base of upper leaf sinuses — U-shaped Tooth at petiole sinus — Absent Density of prostrate hairs between veins on lower surface of blade — Very sparse Density if erect hairs between veins on lower surface of blade — Absent Density of prostrate hairs on main veins on lower surface of blade — Sparse Density of erect hairs on main veins on lower surface of blade — Sparse Density of prostrate hairs on main veins on upper surface of blade — Sparse Density of erect hairs on main veins on upper surface of blade — None Autumn coloration of leaves — Grey-Yellow; 161A, B, 163A, B, 162A.
   Upper surface.—Color — Green; 137A, B Anthocyanin coloration of main veins — Absent Surface appearance — Semi-glossy — Dull Blistering surface of blade — Weak — Medium.
   Lower surface.—Color — Green; 146B Anthocyanin coloration of main veins (lower surface) — Absent Glossiness — Weak Surface texture — Rugose Surface appearance — Dull.
   Petiole.—Length — About 10.5 cm Length of petiole compared to middle vein — Slightly shorter Density of prostrate hairs on petiole — Sparse Density of erect hairs on petiole — None.
   Buds.—Bud fruitfulness — Basal: Mostly fruitful Position of first fruitful shoot on previous season cane — 1^{st} to 2^{nd} node Time of bud burst — Medium; Mar. 10, 2010.
Flowers:
   General.—Flower sex — Hermaphrodite Length of first inflorescence — Medium; About 21.3 cm long by 12.3 cm wide Position of first flowering and fruiting node — 3^{rd}-4^{th} node (current season growth) Number of inflorescence per flowering shoot — 1.1 to 2 Time of bloom — Medium — Late as compared with similar varieties in the growing area of Delano, Calif. Date of full bloom — May 18, 2010.
Fruit:
   General.—Ripening period — Late; Approximately Sep. 16, 2010 Use — Fresh market Keeping quality — Excellent Resistance to — insects: Average typical of Vitis vinifera species Diseases: Average typical of Vitis vinifera species Refractometer test — solid-sugar: About 18.8 Brix Brix/acid — About 72.3 Titratable acidity — About 0.26 Juice pH — About 4.0.
   Cluster.—Mature cluster length (peduncle excluded) — About 35.8 cm Mature cluster width — About 14.8 cm Mature cluster weight — About 1384 g Bunch density — Medium Number of berries — About 236 Form — Conical.
   Peduncle.—Lignification of peduncle — Weak Length of peduncle — Medium Approximately 5.0 cm.
   Berry.—Uniformity of size — Slightly Variable Single berry weight — About 8.9 g natural; to about 9.7 g when treated with gibberellic acid Shape — Narrow elliptic Seeds — Absent Cross section — Circular Berry dimensions — longitudinal axis: About 31.1 mm Horizontal axis: About 21.6 mm Berry firmness — Medium Particular flavor — Neutral Bloom (cuticular wax) — Medium Berry separation from pedicel — Medium Skin color (without bloom) — Yellow-green; N144D, 145A, 151A.
   Skin.—Thickness — Medium Texture — Medium Reticulation — Absent Tenacity — Tenacious to flesh.

What is claimed:

1. A new and distinct variety of grapevine as herein illustrated and described
